(19) United States
(12) Patent Application Publication
(10) Pub. No.: US 2009/0159458 A1
(43) Pub. Date: Jun. 25, 2009

(54) Method for Determination of Test Substance

(75) Inventors: Eiichi Tamiya, Ishikawa (JP); Naoki Nagatani, Okayama (JP); Teruko Yuhi, Ishikawa (JP); Kagan Kerman, Saskatchewan (CA); Koutarou Idegami, Ishikawa (JP); Miyuki Chikae, Ishikawa (JP)

Correspondence Address: KANESAKA BERNER AND PARTNERS LLP, 1700 DIAGONAL RD, SUITE 310, ALEXANDRIA, VA, 22314-2848, US

(73) Assignees: Bio Device Technology Ltd., Ishikawa (JP); Japan Advanced Institute of Science and Technology, ISHIKAWA (JP)

(21) Appl. No.: 12/226,060

(22) PCT Filed: Mar. 29, 2007
(86) PCT No.: PCT/JP2007/056992

§ 371 Date: Feb. 12, 2009

(30) Foreign Application Priority Data
Apr. 7, 2006 (JP) 2006-106583
Sep. 28, 2006 (JP) 2006-265183

(51) Int. Cl. G01N 27/416; G01N 33/543

(52) U.S. Cl. 205/704

(57) ABSTRACT

[Problems] A highly sensitive and accurate determination of a test substance is achieved without the need of complicated procedures.

[Means for solving the Problems] The presence or absence or the concentration of a test substance 3 can be determined by gathering around the surface of a working electrode 1 metal microparticles 5 in an amount corresponding to the amount of the test substance 3 contained in a test solution, oxidizing the metal microparticles 5 electrochemically, measuring the value of a current produced by electrochemically reducing the oxidized metal, and determining the presence or absence or the concentration of the substance 3 based on the current value. It is preferred that the electrochemical oxidation of the metal microparticles 5 be conducted while maintaining the potential of the working electrode 1 at a value equal to a potential employed for the electrochemical oxidization of the metal microparticles 5.

### TECHNICAL FIELD

The present invention relates to a method for the determination of a test substance using an electrochemical technique.

The immunoassay utilizing an antigen-antibody reaction has been known as one of the methods for simply determining a small amount of a substance in a test solution with high sensitivity. As the immunoassay, the ELISA method has been adopted in a wide range of fields, which method uses an antibody labeled with an enzyme to obtain a chromogenic or light-emitting signal resulting from an enzymatic reaction, thereby performing the detection or concentration measurement of a test substance. However, since the ELISA method necessitates an optical system when detecting the chromogenic or light-emitting signal, it is necessary to use a large-sized measuring machine. In addition, when performing accurate qualification, the operation of converting measurement results of the chromogenic signal into electrical signals has to be performed. Thus, a complicated treatment has to be performed.

So, in the immunoassay using a general-purpose labeled substance, such as a chromogenic label or a fluorescent label, a method utilizing an electrochemical measuring method during the detection has been proposed. Since the device used for the electrochemical measurement can be made small as compared with equipment used for the ELISA, the miniaturization of measuring equipment and the enhancement of detection sensitivity are expected to be compatible. In Patent Document 1, for example, metal microparticles are dissolved using a chemical treatment, an electrochemical measurement is then made, and a qualitative or quantitative analysis of a test substance is performed based on a peak current induced by oxidation of the metal microparticles obtained.

Incidentally, described in Non-Patent Document 1 is a method for measuring a reduced peak-current of a gold colloid in a solution or an antibody labeled with a gold colloid.

### Patent Document 1: JP2004-512496A

### Non-Patent Document 1: Bioelectrochemistry and Bioenergetics 38 (1995) 389-395

### DISCLOSURE OF THE INVENTION

### Problems the Invention Intends to Solve

In Patent Document 1, however, since the step of completely dissolving the metal microparticles by means of a chemical treatment using a solution has to be taken preparatory to the electrochemical measurement, an inconvenient problem is entailed in that the measuring operation becomes bothersome. Though, in Patent Document 1, the electrochemical measurement is used to measure the value of a current induced by oxidation of the metal microparticles, since the oxidation current value obtained includes the target current resulting from the metal microparticles and a relatively large quantity of noises, like a current, resulting from the antibody used for the measurement or from the foreign substances in the measurement solution, false detection may possibly be induced. Furthermore, Non-Patent Documents 1 merely examines the relationship between the concentration of the metal colloid in the solution and the reduced current and has no description therein concerning the quantification of the test substance in the solution.

The present invention has been proposed in view of the conventional real nature and the object thereof is to provide a method for accurately measuring a test substance with high sensitivity.

### Means for solving the Problems

To attain the above object, the present invention provides a method for determining a test substance, comprising the steps of gathering in the neighborhood of the surface of a working electrode metal microparticles in an amount corresponding to the amount of the test substance contained in a test solution, oxidizing the metal microparticles electrochemically, measuring the value of a current induced by electrochemically reducing the oxidized metal microparticles, and determining the presence or absence or the concentration of the test substance based on the current value.

In the test substance-determining method as described above, mutual interaction of biological materials like an antigen-antibody reaction, for example, is first utilized to gather metal microparticles in an amount corresponding to the amount of the test substance, the metals constituting the metal microparticles are electrochemically oxidized, and then the value of a reduced current induced when reducing the oxidized metals is measured. Since the reduced current intensity obtained here represents the amount of the metals gathered in the neighborhood of the working electrode, based on this, quantification or detection of the test substance is realized. Here, it is important to perform the electrochemical oxidation of the metal microparticles in the state of the metal microparticles being gathered in the neighborhood of the surface of the working electrode. With this, all the metal microparticles having pertained to the reaction with the test substance can be involved in giving and receiving ions to and from the surface of the working electrode and, as a result, it is realized to measure the test substance with high sensitivity.

Since the noise included in the reduced current value obtained in consequence of the above measurement is smaller than that obtained by the conventional electrochemical measurement, the present invention makes it possible to accurately detect the test substance. In addition, since the oxidation of the metal microparticles can easily be realized by means of the potential control of the working electrode, the complicatedness of the determination operation can be kept to the minimum as compared with the case of the oxidation by a chemical treatment, for example.

### EFFECTS OF THE INVENTION

According to the present invention, it is possible to realize highly sensitive and accurate determination of the test substance contained in the test solution with the simple operation without the need of the large-sized measuring equipment as used in the detection step of the ELISA, for example.

### BRIEF DESCRIPTION OF THE DRAWINGS

FIG. 1 includes schematic cross-sectional views illustrating the principal part of the first embodiment, (a) showing a working electrode having a primary antibody fixed thereto, (b) showing an antigen-antibody reaction, (c) showing oxidation of metal microparticles gathered in the neighborhood of the surface of the working electrode and (d) showing the measurement of a reduced current.

FIG. 2 includes schematic cross-sectional views illustrating the principal part of the second embodiment, (a) showing an antigen-antibody reaction, (b) showing trapping of magnetic microparticles, (c) showing supply of the magnetic microparticles to the electrode surface, (d) showing oxidation of the microparticles gathered in the neighborhood of the surface of the working electrode and (e) showing the measurement of a reduced current.

FIG. 3(a) is a schematic plan view showing a strip for immunochromatography and (b) a schematic side view thereof.

FIG. 4 includes schematic cross-sectional views illustrating the principal part of the third embodiment, (a) showing a strip for immunochromatography having a primary antibody fixed thereto, (b) showing an antigen-antibody reaction, (c) showing oxidation of metal microparticles gathered in the neighborhood of the surface of the working electrode and (d) showing measurement of a reduced current.

FIG. 5 includes schematic cross-sectional views illustrating the principal part of the fourth embodiment, (a) showing a working electrode having a primary antibody fixed thereto and a counter electrode, (b) showing an antigen-antibody reaction, (c) showing deposition of metals on the surface of the working electrode, (d) showing oxidation of metal microparticles gathered in the neighborhood of the surface of the working electrode and (e) showing measurement of a reduced current.

FIG. 6 is a plane view showing a printed electrode device used in Experiments 1 to 3.

FIG. 7 is a diagram showing the relationship between the working electrode potential and the current value.

FIG. 8 is a diagram showing the relationship between the hCG concentration and the current value.

FIG. 9 is a diagram of the results of Experiment 2 showing the relationship between the hCG concentration and the current value.

FIG. 10 is a diagram of the results of Experiment 3 showing the relationship between the working electrode potential and the current value.

FIG. 11 is a plan view showing a printed electrode device used in Experiment 4.

FIG. 12 includes photographs showing the surfaces of the counter electrode before and after application of voltage and the surface of the working electrode, (a) showing the counter electrode before the application of voltage, (b) showing the counter electrode after the application of voltage and (c) showing the edge part of the working electrode after the application of voltage.

FIG. 13 is a diagram showing comparison results in cyclic voltammetry between the cases where a hydrochloric acid and an aqueous potassium chloride solution are used as respective measurement solutions.

FIG. 14 is a diagram showing comparison results in differential pulse voltammetry between the cases where a hydrochloric acid and an aqueous potassium chloride solution are used as respective measurement solutions.

FIG. 15 is a characteristic diagram showing the comparison between the cases where an aqueous saturated potassium chloride solution and an aqueous 1M potassium chloride solution are used as respective measurement solutions.

FIG. 16 is a characteristic diagram showing the results of study on optimum particle diameters of metal colloids.

FIG. 17 is a diagram showing the relationship between the hCG concentration and the current value.

FIG. 18(a) is a diagram showing the relationship between the hCG concentration and the reduced current value according to the determination method of the present invention and (b) a diagram showing the relationship between the hCG concentration and the absorbance according to the ELISA method.

FIG. 19(a) is a characteristic diagram showing the relationship between the working electrode potential and the oxidation current value and (b) a characteristic diagram showing the relationship between the working electrode potential and the reduced current value.

FIG. 20 is a characteristic diagram showing the relationship between the oxidation potential application time and the reduced current value when the applied potentials have been set at 1.2 V, 1.4 V and 1.6 V, respectively.

FIG. 21 is a characteristic diagram showing the relationship between the oxidation potential application time and the reduced current value when the hCG concentrations in the test solutions have been set to be 62 pg/ml, 620 pg/ml and 62 ng/ml, respectively.

FIG. 22 is a characteristic graph showing the results of study on concentrations in the case of using a hydrochloric acid as a measurement solution.

### EXPLANATION OF REFERENCE NUMERALS

1: working electrode, 2: primary antibody, 3: test substance (antibody), 3: secondary antibody, 5: metal microparticle, 11: magnetic microparticle, 12: container, 13: reaction solution, 14: magnet, 15: solution for electrochemical measurement, 21: strip for immunochromatography, 22: membrane, 23: determination part, 24: control part, 31: counter electrode, 32: substrate, 41: printed electrode, 42: working electrode, 43: counter electrode, 44 reference electrode, 45: insulating support, 46: insulating layer, 51: planar-type printed electrode device, 52: insulating coat, 53: working electrode, 54: counter electrode, 55: reference electrode, and 56: insulating support substrate.

### BEST MODE FOR CARRYING OUT THE INVENTION

The test substance-determining method of the present invention will be described hereinafter in detail with reference to the drawings.

### First Embodiment

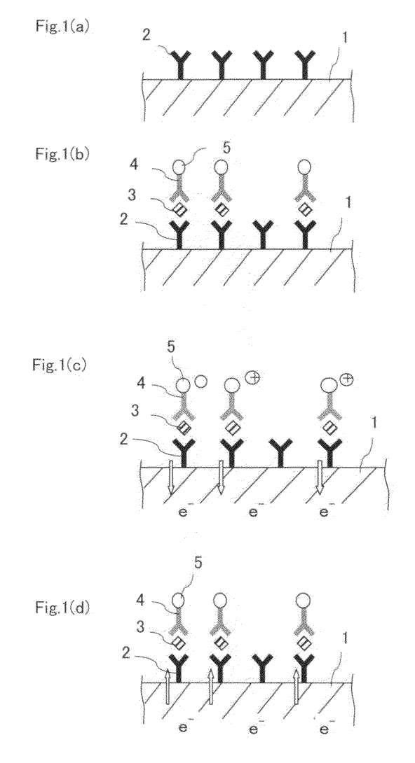

In the first embodiment, two kinds of specific binding substances relative to a test substance, one of which (first binding substance) is fixed to a working electrode and the other of which (second binding substance) is labeled with metal microparticles to constitute a labeled body, are prepared. To be specific, first, primary antibodies 2 that are the first binding substances relative to the test substances 3 are fixed to the surface of a working electrode 1 to be used in electrochemical measurement (FIG. 1(a)). The electrode surface is blocked for the purpose of preventing nonspecific adsorption. Then, secondary antibodies 4 prepared as the second binding substances for recognizing different sites on the test substances 3 are labeled with metal microparticles 5 to constitute labeled bodies.

A test solution containing the labeled bodies and an unknown amount of the test substances 3 is supplied onto the surface of the working electrode 1, thereby bringing the test solution into contact with the primary antibodies to make an antigen-antibody reaction on the working electrode 1. The labeled bodies are bound to the primary antibodies 2 via the test substances 3 to go into a state in which the metal microparticles 5 in an amount corresponding to the concentration of the test substances 3 have been gathered in the neighborhood of the working electrode 1 (FIG. 1(b)).

It is noted here that all substances, such as biological materials and synthesized materials, can be used as the test substances. The binding substances (first and second binding substances) bound specifically to the test substances are suitably selected depending on the nature of the test substances. Though the present embodiment utilizes specific binding between an antigen and an antibody in order to gather the metal microparticles in an amount corresponding to the amount of the test substances contained in the test solution, this combination is not limitative insofar as two substances can specifically be bound to each other. For example, specific binding of nucleic acid-nucleic acid, nucleic acid-nucleic acid-binding protein, lectin-sugar chain or receptor-ligand may be utilized. In each of these combinations, the order of the test substance the specifically binding substance may be reversed.

Though the metal microparticles 5 used as the labeled substances are not particularly restricted, microparticles of gold, platinum, silver, copper, rhodium, palladium, colloidal particles or quantum dots thereof, for example, can be used. Among other metal microparticles enumerated above, gold microparticles having a particle diameter of 10 nm to 60 nm, particularly around 40 nm, are preferably used.

The antigen-antibody reaction is made, the surface of the working electrode 1 is washed when necessary, and then a solution for electrochemical measurement, for example, and the working electrode 1 are brought to a mutually contacting state. As the means for bringing the solution into contact with the working electrode 1, optional means, such as putting drops of the solution onto the surface of the working electrode 1 and immersion of the working electrode 1 in the solution, can be adopted.

Next, the metal microparticles are electrochemically oxidized. For example, the potential of the working electrode 1 relative to a reference electrode is retained for a predetermined time at a level at which the metal microparticles 5 can electrochemically be oxidized. As a result, the metal microparticles 5 gathered in the neighborhood of the surface of the working electrode 1 can completely be oxidized (FIG. 1(c)). At this time, though not shown, the counter electrode and reference electrode are also brought to a state of contact with the solution.

The presence or absence or the concentration of the test substance is determined based on the value of a peak current induced when reducing the metal microparticles oxidized electrochemically (FIG. 1(d)). To be specific, the potential of the working electrode 1 is varied in the negative direction, and current changes with the potential variation are measured. The variation in electrode potential in the negative direction allows the potential control to reduce the metal oxidized and eluted and flow the reduced current, which is measured. Since the more the amount of the test substances in the test solution and the more the amount of the metal particles gathered in the neighborhood of the working electrode 1, the larger the reduced current intensity, the qualification or detection of the test substances is realized based on this relational fact. By obtaining the relation in advance between the reduced current value and the already known concentration of the test substance and comparing the reduced current value measured with the relation obtained, the concentration of the test substances can be obtained. Furthermore, also the presence or absence of the test substances in the test solution can be detected from the reduced current value obtained.

As the solution used in the potential control and electrochemical measurement of the working electrode 1, an acidic solution is preferably used because it can easily oxidize the metal microparticles 5 electrochemically. The acidic solution may suitably be selected in accordance with the kind of the metal microparticles 5. For example, an aqueous solution containing hydrochloric acid, nitric acid, acetic acid, phosphoric acid, citric acid, sulfuric acid, etc. can be used. In view of ease of electrochemical oxidation of the metal microparticles, use of a 0.05N to 2N aqueous solution of hydrochloric acid is preferable, and use of a 0.1N to 0.5N aqueous solution of hydrochloric acid is more preferable.

On the other hand, as the solution used in the potential control and electrochemical measurement of the working electrode 1, a neutral solution containing chlorine can also be used, besides the acidic solution. Use of the neutral solution containing chlorine enables a large amount of current changes to be obtained as compared with use of the acidic solution and, as a result, more highly sensitive measurement is achieved. Furthermore, in the case of using the acidic solution, the skirt of the reduced peak on the side of low potential, for example, is raised to make the peak shape asymmetry, and a noise may possibly be generated in the vicinity of 0.1 V, for example. On the other hand, since use of the neutral solution containing chlorine enables the skirts of the reduced peak to be flat and the generation of a noise to be suppressed, simple detection of the reduced peak intensity can be attained. Furthermore, it can avoid use of an acidic or alkali solution difficult to handle and make the measurement operation safe and simple. KCl, NaCl and LiCl used as the neutral solution containing chlorine can bring about the above effect. Use of KCl in particular gives rise to a greater effect.

The potential of the working electrode 1 in oxidizing the metal microparticles 5 is set at a level at which the metal microparticles 5 can be oxidized. Specifically, although it is necessary to appropriately set the potential of the working electrode 1 at an optimal level in accordance with the kind of the metal microparticles 5, the potential is preferably set in the range of +1 V to +2V relative to a silver/silver chloride reference electrode. The potential of the working electrode 1 falling in the above range enables the metal microparticles 5 gathered in the neighborhood of the surface of the working electrode 1 to be completely oxidized and eluted and the detection sensitivity of the test substance 3 to be infallibly enhanced. When the potential of the working electrode 1 falls short of the above range, there is a possibility of the peak of the reduced current failing to emerge during the measurement. To the contrary, when it exceeds the above range, the metal particles oxidized are spread by electrophoresis to lower the concentration of the oxides in the neighborhood of the working electrode 1, thereby possibly making the peak of the reduced current small. More preferable range thereof is in the range of +1.2 V to +1.6V.

Raised as concrete means for electrochemically oxidizing the metal microparticles is to retain the potential of the working electrode 1 for a prescribed time at a level at which the metal microparticles are oxidized. The operation of retaining the potential for the prescribed time is a preferable method because the metal microparticles can sufficiently be oxidized. When applying a potential capable of electrochemically oxidizing the metal microparticles to the working electrode, it may be adopted to vary the potential of the working electrode with time by means of cyclic voltammetry, for example, besides the method of retaining the potential of the working electrode at the prescribed level as described above. When varying the potential of the working electrode with time, it is preferred that the potential of the working electrode be varied in the potential range in which the metal microparticles are oxidized (in the range of +1 V to +2 V relative to the silver/silver chloride reference electrode, for example). Furthermore, when oxidizing the metal microparticles, it may be adopted to apply a potential capable of electrochemically oxidize the metal microparticles to the working electrode a plurality of times.

When using gold microparticles having a particle diameter of 10 nm to 60 nm as the metal microparticles, it is preferred in electrochemically oxidizing the gold microparticles that the potential of the working electrode be in the range of +1.2 V to +1.6 V relative to the silver/silver chloride reference electrode.

In oxidizing the metal microparticles 5 sufficiently, it is necessary to draw attention to impartation of an optimum amount of electric charges in accordance with the amount of the metal microparticles 5. Since the amount of electric charges is the value obtained by integrating the current, when the potential applied to the working electrode 1 is a relatively low value, it is necessary to apply the low potential for a long period of time in order to sufficiently oxidize the metal microparticles 5. On the other hand, when the potential applied to the working electrode 1 is a relatively high value, the time required to oxidize the metal microparticles 5 may be small.

When the time for retaining the potential of the working electrode 1 at a level at which the metal microparticles 5 are electrochemically oxidized is set to be one second or more, it is possible to sufficiently oxidize the metal microparticles and infallibly enhance the detection sensitivity. On the other hand, even when the potential-applying time is set to be 100 seconds or more, the current value obtained is unchanged. Therefore, the time is preferably in the range of one second to 100 seconds. The more preferable potential-applying time is in the range of 40 seconds to 100 seconds.

As a method for measuring the current induced when reducing the oxidized metal electrochemically, the voltammetry, such as differential pulse voltanmmetry and cyclic voltanmmetry, amperometry and chronometry can be cited.

In the first embodiment as described above, since the antigen-antibody reaction is made on the working electrode to gather the metal microparticles in the neighborhood of the surface of the working electrode and since the reduced peak current resulting from the metal microparticles contained in the labeled body is measured, it is possible to simply determine the test substance contained in the test solution with high sensitivity.

### Second Embodiment

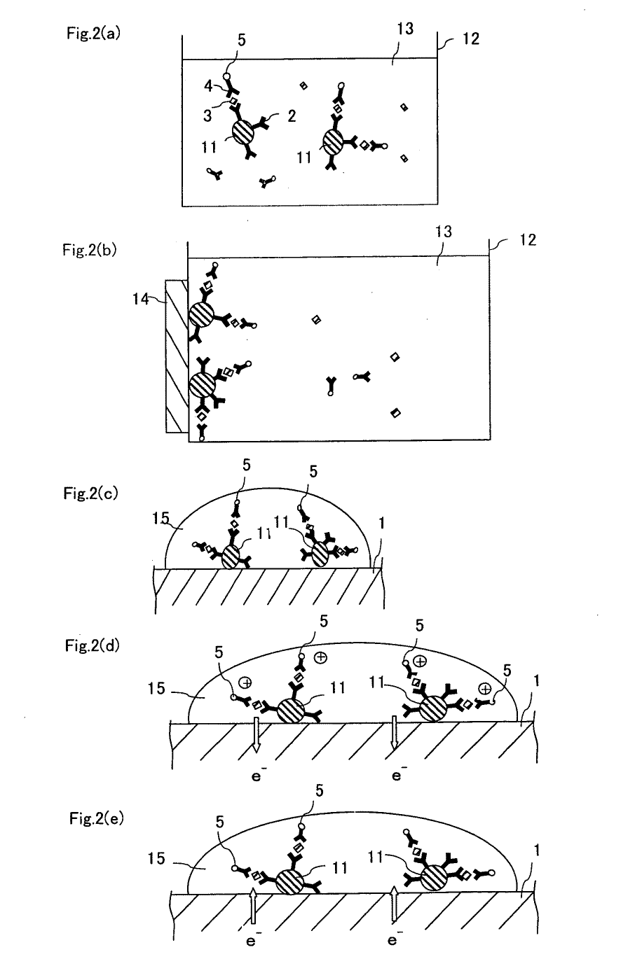

The second embodiment differs from the first embodiment in concrete means for gathering in the neighborhood of the surface of the working electrode the metal microparticles in an amount corresponding to the amount of the test substance in the test solution. To be specific, in gathering the metal microparticles in the amount corresponding to the amount of the test substance in the test solution in the neighborhood of the surface of the working electrode according to the second embodiment, the realization thereof is achieved through the steps of preparing two kinds of binding substances relative to the test substance, one of which (first binding substance) is fixed to the surfaces of magnetic microparticles and the other of which (second binding substance) is labeled with metal microparticles to constitute a labeled body, reacting the magnetic microparticles with the labeled body and gathering the reacted magnetic microparticles on the surface of the working electrode. The second embodiment will be described hereinafter with reference to FIG. 2. Incidentally, in each of the embodiments described herein below, the descriptions that have already been made with respect to the first embodiment will be omitted.

First, primary antibodies 2 are fixed to the surfaces of magnetic microparticles 11 as first binding substances that are specifically bound to test substances 3. On the other hand, secondary antibodies 4 serving to recognize a site different from the sites of the primary antibodies having been fixed to the magnetic microparticles 11 are labeled with metal microparticles to constitute labeled bodies.

Next, a reaction solution 13 is prepared in a given container 12. The reaction solution 13 is a mixture of the magnetic microparticles 11 having the primary antibodies fixed thereto, secondary antibodies 4 labeled with the metal microparticles 5 and a test solution containing the test substances having an unknown concentration and is incubated for a prescribed period of time to make an antigen-antibody reaction on the magnetic microparticles 11. As a result, the labeled bodies are bound to the magnetic microparticles 11 via the test substances 3 (FIG. 2(a)).

Subsequently, a magnet 14 is used to separate the magnetic microparticles from the reaction solution 13 (FIG. 2(b)). Thereafter, the separated magnetic microparticles 11 are suspended in a solution for electrochemical measurement.

Next, the metal microparticles 5 are gathered in the neighborhood of the surface of the working electrode 1 (FIG. 2(c)). To be specific, the magnetic microparticles 11 to which labeled bodies have been bound are suspended in the solution for electrochemical measurement and a suspending solution is then supplied as putting drops thereof on the surface of the working electrode 1. Thereafter, the resultant solution is left at rest for a prescribe period of time, for example, to precipitate the magnetic microparticles 11. A state in which the metal microparticles 5 have been gathered in the neighborhood of the surface of the working electrode 1 is consequently obtained. Otherwise, by disposing a magnet on the rear surface of the working electrode 1 to magnetically attract the magnetic microparticles 11 to the front surface of the working electrode 1, the time until the metal microparticles 5 having been bound to the magnetic microparticles 11 are gathered in the neighborhood of the working electrode 1 can be shortened.

The subsequent steps are the same as those in the first embodiment as described above. That is to say, the metal microparticles 5 are oxidized electrochemically. Preferably, the potential of the working electrode 1 relative to the reference electrode is retained for a prescribed period of time at a level at which the metal microparticles 5 are oxidized electrochemically. As a result, the metal microparticles 5 having been gathered in the neighborhood of the working electrode 1 are completely oxidized (FIG. 2(d)). At this time, though not shown, the counter electrode and reference electrode are also brought to a state of contact with the solution.

The presence or absence or the concentration of the test substance is determined on the basis of the value of a peak current induced when reducing the oxidized metal after the electrochemical oxidation (FIG. 2(e)). To be concrete, the potential is varied in the negative direction and changes of current with the potential variation are measured. In this way, it is possible to determine the concentration of the test substance or the presence or absence of the test substance similarly to the first embodiment.

In addition, in the present embodiment, since the primary antibodies 2 are fixed to the magnetic microparticles 11 capable of being suspended in the reaction solution to trap the test substance 3, the efficiency of the reaction between the test substance 3 and the labeled bodies can be heightened as compared with the case where the first antibodies 2 are fixed to the surface of the working electrode 1.

Furthermore, by the use of the magnetic microparticles 11 capable of magnetic separation, the amount of the solution for electrochemical measurement required for suspending the magnetic microparticles 11 can be reduced. That is to say, since the magnetic microparticles 11 (metal microparticles 5) can exist in the solution for electrochemical measurement in a high concentration, the detection sensitivity can further be enhanced.

### Third Embodiment

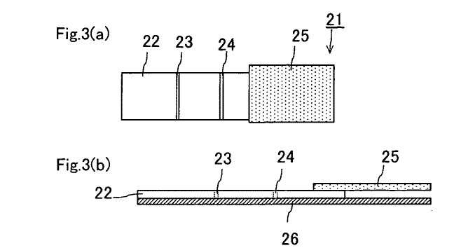

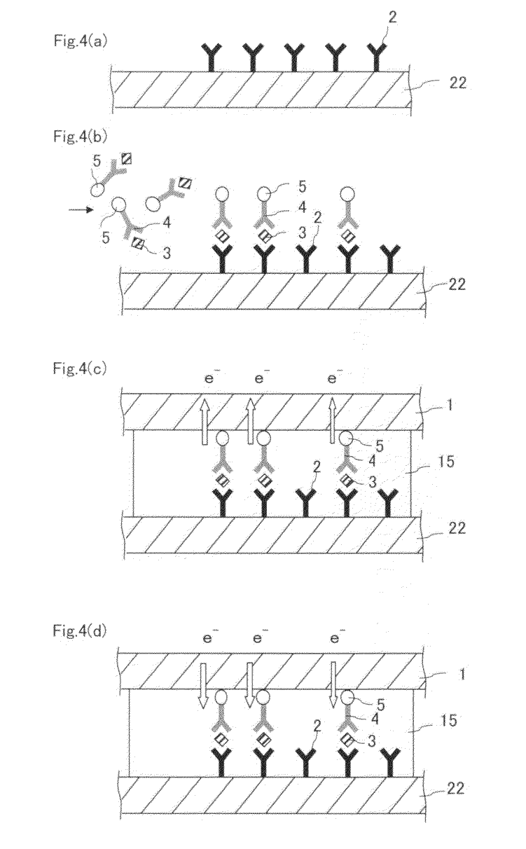

The third embodiment differs from the first embodiment in concrete means for gathering in the neighborhood of the surface of the working electrode the metal microparticles in an amount corresponding to the amount of the test substance in the test solution. That is to say, in the third embodiment, in gathering the metal microparticles in the amount corresponding to the amount of the test substance in the test solution in the neighborhood of the surface of the working electrode, the realization thereof is attained by the steps of preparing two kinds of specific binding substances relative to the test substance, one of which (first binding substance) is fixed to a determination part of a strip for immunochromatography and the other of which (second binding substance) is labeled with the metal microparticles to constitute labeled body, developing the test solution and labeled body on the strip and then disposing the strip and the surface of the working electrode face to face. An example adopting the immunochromatographic method will be described hereinafter with reference to FIG. 3 and FIG. 4.

Though the structure of a strip used for the immunochromatographic analysis is not particularly restricted, a strip 21 for immunochromatography as shown in FIG. 3, for example, can be used. The immunochromatography strip 21 comprises a reed-shaped membrane 22 formed of nitrocellulose, an absorption pad 25 joined to the downstream side of the membrane 22 and a backing sheet 26 attached to the back surface side of the membrane 22. As shown in FIG. 4(a), first antibodies 2 are fixed to a prescribed region on the surface of the membrane 22 to constitute a determination part 23. Antibodies bound specifically to second antibodies 4 labeled with metal microparticles 5 are fixed to the surface of the membrane 22 downstream of the determination part 23 to constitute a control part 24.

In order to detect test substances contained in a test solution, the test solution is first developed in the same manner as in the ordinary immunochromatographic method. To be specific, the test solution and secondary antibodies 4 labeled with the metal microparticles 5 are mixed and the resultant mixture is absorbed on one end of the immunochromatography strip 21 (on the left side in FIG. 3) to develop the mixture utilizing the capillary phenomenon. When the test substances 3 exist in the test solution, the primary antibody 2 and secondary antibody 4 are bound to the test substance 3 as sandwiching the test substance to consequently trap the metal microparticles 5 in an amount corresponding to the amount of the test substance 3 onto the determination part 23 (FIG. 4(b)). The termination of the development can be found from the color phenomenon from the labeled secondary antibodies trapped onto the control part 24.

Then, at least the determination part 23 of the membrane 22 and the working electrode 1 are caused to overlap each other. As a result, the metal microparticles 5 gathered at the determination part 23 approach and are gathered in the neighborhood of the surface of the working electrode 1 (FIG. 4(c)). In order to reduce the distance between the metal microparticles 5 and the working electrode 1 with exactitude, pressure may be applied after the overlap between at least the determination part 23 of the membrane and the working electrode 1. The pressure application is performed slightly to an extent of bringing the surfaces of the membrane 22 and working electrode 1 into infallible contact with each other.

The gap between at least the determination part 23 of the membrane 22 and the working electrode 1 is filled with a solution 15 for electrochemical measurement. At this time, a counter electrode and a reference electrode are also brought to a state of contact with the solution 15.

The subsequent steps are the same as in the first embodiment. That is to say, the metal microparticles 5 are oxidized electrochemically. Preferably, the potential of the working electrode 1 relative to the reference electrode is retained for a prescribed period of time at a level at which the metal microparticles 5 are electrochemically oxidized. As a consequence, the metal microparticles 5 that have been gathered in the neighborhood of the surface of the working electrode 1 can completely be oxidized.

The presence or absence or the concentration of the test substances is determined based on the value of the peak current induced when reducing the oxidized metal after the electrochemical oxidation (FIG. 4(d)). Specifically, by varying the potential of the working electrode 1 in the negative direction, current changes with the potential variation are measured. In this way, the concentration of the test substances can be obtained in the same manner as in the first embodiment. In addition, the presence or absence of the test substances contained in the test solution can be found from the value of reduced current obtained. Furthermore, highly sensitive and quantitative analysis can be realized without impairing the simplicity of the immunochromatographic analysis.

### Fourth Embodiment

The fourth embodiment will be described herein below. In the first embodiment described above, the antigen-antibody reaction is made, for example, with the first antibodies fixed only to the working electrode as the primary binding substances and only the working electrode as the reaction field. However, in the case of using a planar-type device having a working electrode, counter electrode and reference electrode formed as being printed on the same substrate, the area of the working electrode as the reaction filed is limited depending on the size of the device per se and areas of the counter and reference electrodes. In the determination method described in the first embodiment, therefore, the sensitivity enhancement has its own limits.

In view of the above, in the present embodiment, at least both the working electrode and the counter electrode are used as reaction fields of the antigen-antibody reaction. In addition, the metal microparticles gathered in the neighborhood of the surface of at least the counter electrode are oxidized and eluted, and electrochemically subjected to electrophoresis to deposit the metal microparticles on the surface of the working electrode. Thereafter, the metal microparticles gathered in the neighborhood of the surface of the working electrode are electrochemically oxidized in the same manner as in the first embodiment. As a consequence, since the metal microparticles gathered in regions other than the working electrode are to be subjected to the electrochemical measurement, more amounts of the metal microparticles are efficiently gathered on the surface of the working electrodes to realize further enhancement of the detection sensitivity.

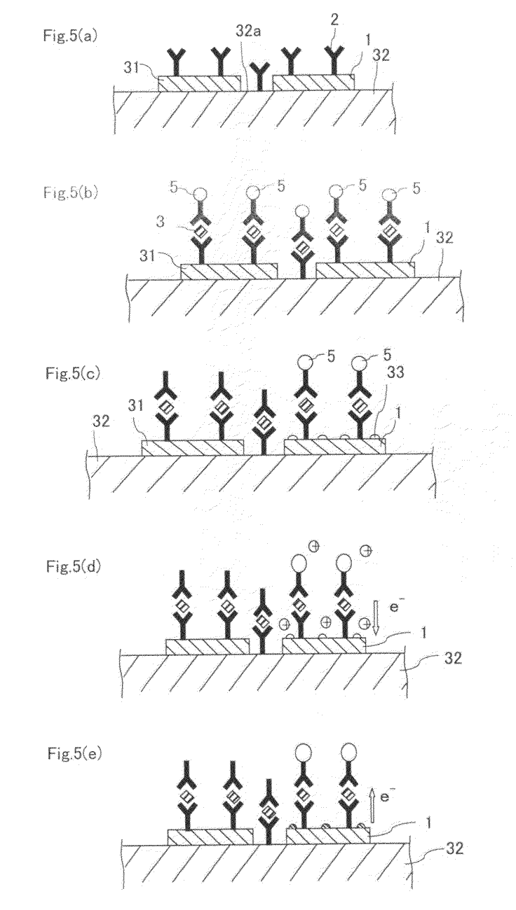

To be specific, as shown in FIG. 5(a)), a planar-type electrode device having a working electrode 1, a counter electrode 31 and a reference electrode (not shown) formed on a single substrate 32 is first prepared. Primary antibodies 2 as the first binding substances relative to the test substances are bound to both the working electrode 1 and the counter electrode 31. In addition, the primary antibody 2 is fixed onto an interelectrode region 32a intervening between the working electrode 1 and the counter electrode 31 on the substrate 32. This further fixation of the primary antibody 2 onto the interelectrode region 32a intervening between the working electrode 1 and the counter electrode 31 can achieve further highly sensitive detection. The surface of the electrode device is blocked for the purpose of preventing nonspecific adsorption. On the other hand, secondary antibodies 4 are prepared as second binding substances for recognizing different sites on the test substances 3 and labeled with the metal microparticles 5 to constitute labeled bodies. Incidentally, insofar as the working electrode, counter electrode and reference electrode are disposed at places close to one another, it is not always necessary for these electrodes to be formed on the same substrate.

Subsequently, a test solution containing the labeled bodies and an unknown amount of the test substances 3 is supplied onto the surfaces of the working electrode 1, counter electrode 31 and interelectrode region 32a to come into contact with the primary antibodies 2, thereby making an antigen-antibody reaction on the working electrode 1, counter electrode 31 and interelectrode region 32a. The labeled bodies are bound via the test substances 3 to the primary antibodies 2 to create a state in which the metal microparticles 5 having a concentration corresponding to that of the test substances 3 have been gathered in the neighborhood of the surfaces of the working electrode 1, counter electrode 31 and interelectrode region 32a (FIG. 5(b)).

Next, the surfaces of the working electrode 1, counter electrode 31 and interelectrode region 32a are washed, as occasion demands, and brought into contact with a solution for electrochemical measurement to make potential control so as to cause the counter electrode 31 to have a positive potential relative to the working electrode 1. As a result, the metal microparticles 5 gathered in the neighborhood of the surface of the counter electrode 31 are oxidized, eluted and subjected to electrophoresis in the solution. The metal microparticles 5 gathered in the neighborhood of the surface of the interelectrode region 32a intervening between the working electrode 1 and the counter electrode 31 are also subjected to electrophoresis in the solution. The metal microparticles 5 having reached the surface of the working electrode 1 are deposited thereon as metals 33. As a result, all the metal particles 5 pertaining to the reaction are gathered in the neighborhood of the surface of the working electrode 1.

In order to elute at least the metal microparticles 5 in the neighborhood of the surface of the counter electrode 31 and deposit these on the surface of the working electrode 1 by the electrophoresis, the potential of the counter electrode 31 relative to the working electrode 1 has to be at a level at which the metal microparticles 5 are oxidized. Specifically, it is preferred that the potential of the counter electrode 31 be in the range of +1 V to +2 V, for example, relative to the working electrode 1 though varied depending on the kind of the measurement solution to be used. With the above range maintained, at least the metal microparticles 5 gathered on the surface of the counter electrode 31 can be eluted with exactitude and subjected to electrophoresis onto the working electrode 1. The potential of the counter electrode 31 relative to the working electrode 1 may be retained for a prescribed period of time at a level at which the metal microparticles 5 are oxidized or may be varied with time within the range of potential in which the metal microparticles are oxidized.

The subsequent steps are the same as in the first embodiment. That is to say, the metal microparticles 5 that have been gathered in the neighborhood of the surface of the working electrode 1 are oxidized electrochemically. As a result, the metal microparticles 5 that have been gathered in the neighborhood of the surface of the working electrode 1 are completely oxidized (FIG. 5(d)). At this time, the metals 33 that have been deposited on the surface of the working electrode 1 are also oxidized. Next, the peak current induced when reducing the oxidized metals is measured and, based on the measured peak current, the concentration of the test substances is examined (FIG. 5(e)). To be concrete, by varying the potential in the negative direction, for example, current changes with the potential variation are measured. By varying the electrode potential in the negative direction, since the reduced current in consequence of the reduction of the metals oxidized (eluted) by the potential control flows, it is measured. The relationship between the reduced current value and the test substances having known concentrations is measured in advance and is compared with the reduced current value obtained this time to enable the concentration of the test substances to be measured. Furthermore, the presence or absence of the test substances contained in the test solution can be found from the reduced current value thus obtained.

As described in the foregoing, according to the present embodiment, since at least the counter electrode 31 besides the working electrode 1 is also utilized as the reaction field and since the metal microparticles 5 gathered in the neighborhood of the surface of at least the counter electrode 31 are transferred onto the surface of working electrode 1, the reduced current of each of all the metal microparticles 5 in the labeled bodies pertaining to the reaction can be measured to enable realization of further high sensitivity as compared with the case of using only the surface of the working electrode 1 as the reaction field. In addition, since the electrophoresis of the metal microparticles 5 gathered on the surface of at least the counter electrode 31 onto the surface of the working electrode 1 can be achieved through the simple operation of controlling the potentials of the working electrode 1 and counter electrode 31, no mechanical structure for stirring the solution, for example, is required. Therefore, high sensitivity can be realized without modifying the structure on the electrode device side through the simple operation.

In the above description, the method for gathering the metal microparticles in the amount corresponding to the amount of the test substances in the test solution utilizing the non-competitive reaction has been exemplified as the method for gathering the metal microparticles in the amount corresponding to the amount of the test substances. However, it does not matter to adopt a method for gathering the metal microparticles in the amount corresponding to the amount of the test substances in the test solution utilizing the competitive reaction.

### EXAMPLES

Examples of the present invention will be described hereinafter with reference to experimental results.

(Experiment 1)

In this experiment, it was tried to determine human Chorionic Gonadotropin (hCG) diluted with a PBS (Phosphate Buffer Solution) using a printed electrode having a primary antibody (anti-hCG antibody) fixed to the surface of a working electrode. The hCG is a kind of markers for pregnancy diagnosis. As a secondary antibody labeled with gold colloid, an anti-hαS antibody labeled with gold colloid was used.

1. Fixation of Antibody to Working Electrode:

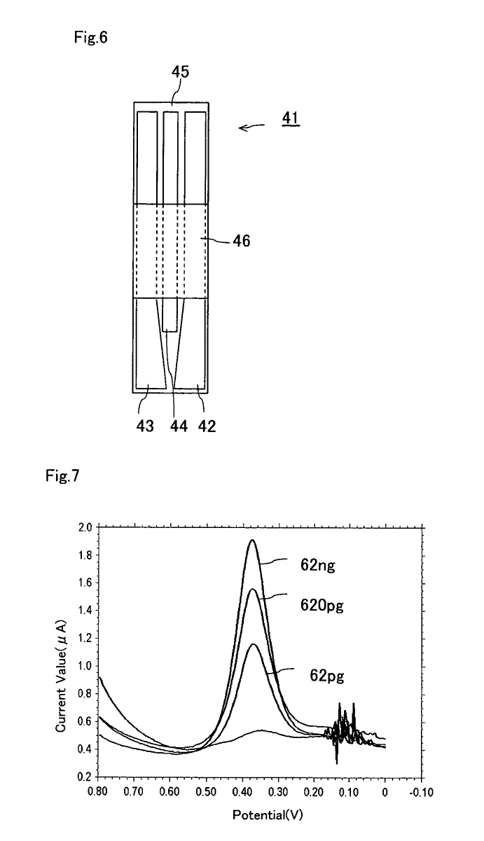

As an electrode device for test substance determination, the planar-type printed electrode device 41 (4 mm in width and 12 mm in length) as shown in FIG. 6 was used. The printed electrode device 41 has a working electrode 42 and a counter electrode 43 both formed of carbon paste, a lead wire (not shown) formed of carbon paste and a reference electrode 44 formed of silver/silver chloride that were all disposed on an insulating support 45. Part of the surfaces of the working electrode 42, counter electrode 43 and reference electrode 44 was insulated with an insulating layer to prescribe respective effective electrode areas.

The working electrode on which 2 μl of drops of an anti-hCG antibody (first antibody) solution adjusted to have a concentration of 100 μg/ml had been put was left at rest for 12 hours in a dark cold place kept at 4° C. to fix the anti-hCG antibody onto the surface of the working electrode. The printed electrode device was washed with a PBS and then blocked with 0.1% cattle serum albumin.

2. Determination of Test Substance:

Solutions were adjusted to have hCG concentrations of 62 pg/ml, 620 pg/ml and 62 ng/ml, respectively, through dilution of hCG as the test substances with a PBS (Phosphate Buffer Solution). The blocked working electrode having antibodies fixed thereto, on which 2 μl of drops of the above solutions had been put, was left at rest for 30 minutes at room temperature to make an antigen-antibody reaction. Thereafter, the printed electrode device was washed with a PBS.

Next, the thus treated working electrode on which 2 μl of drops of a solution of hαS antibody had been put was left at rest at room temperature for 30 minutes to make an antigen-antibody reaction. Thereafter, the printed electrode device was washed with a PBS.

After the washing treatment, 30 μl of drops of an aqueous 0.1N hydrochloric acid solution were put on the thus treated printed electrode device so as to completely cover the overall surfaces of the working electrode, reference electrode and counter electrode, and the potential of the working electrode relative was retained at +1.2 V relative to the reference electrode formed of silver/silver chloride. The retaining time was set to be 40 seconds.

Next, differential pulse voltammetry was used to vary the potential of the working electrode from 0.8 V to −0.1 V, and current changes with the potential variation were measured. The voltammetry conditions included a potential increment of 0.004 V, a pulse amplitude of 0.05 V, a pulse width of 0.05 S and a pulse period of 0.2 S. A characteristic diagram showing changes of current relative to potential is shown in FIG. 7. As shown in FIG. 7, current peaks with the reduction of gold are found in the vicinity of +0.4 V.

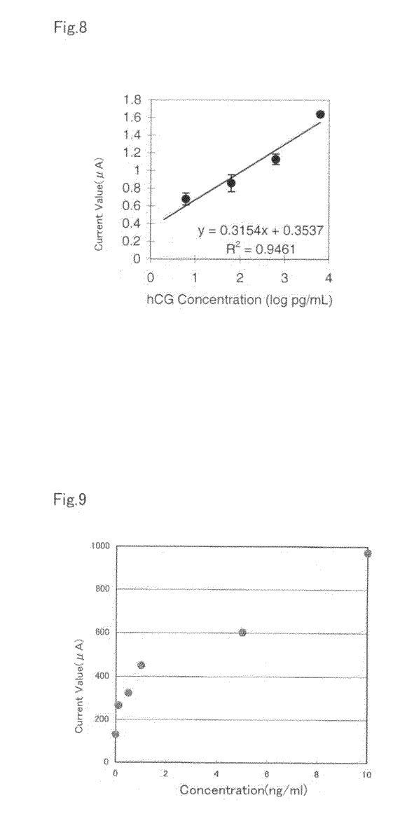

In addition, the relationship between the hCG concentration of the test solution and the current value was shown in FIG. 8. A tendency was found from FIG. 8, in which the higher the hCG concentration, the larger the current value. This involved that the amounts of the antigen (hCG) reacted with the primary antibody (hCG antibody) on the surface of the working electrode and the secondary antibody labeled with gold colloid (hαS antibody labeled with gold colloid) were increased and that the amount of gold colloid reduced on the surface of the working electrode was consequently increased.

(Experiment 2)

Though the immunochromatographic method using metal microparticles as coloring reagents is at an advantage in enabling the presence or absence of test substances to be visually judged with ease, it is unsuitable for quantitative analysis. Though a method that optically measures a determination part of a strip for immunochromatography to obtain the concentration is conceivable, it can be said that good detection sensitivity can be obtained. In view of the above, the present experiment applied a measurement method utilizing the electrochemical measurement of the present invention to an ordinary immunochromatographic analysis using the strip (4 mm in width and 30 mm in length) for hCG-detecting immunochromatography as shown in FIG. 3. In the strip, anti-hCG antibodies and anti-hαS antibodies were fixed to the determination part and the control part, respectively.

Solutions were adjusted to have hCG concentrations of 0.1 ng/ml, 0.5 ng/ml, 1 ng/ml, 5 ng/ml and 10 ng/ml, respectively, through dilution of hCG as the test substances with a PBS.

A gold colloid-labeled hαS antibody was mixed with each of the solutions and the mixture was absorbed on one end of the strip and developed. After completion of the development, the strip was dried. Then, an aqueous 0.1N hydrochloric acid solution was caused to infiltrate the strip and the resultant strip and the printed electrode were caused to overlap each other so that the working electrode of the printed electrode might be brought into contact with determination part as shown in FIG. 6. Incidentally, in this experiment, no primary antibody was fixed to the surface of the working electrode.

Next, the potential of the working electrode was retained at +1.5 V relative to the reference electrode. The retaining time was set appropriately in the range of 30 seconds to 100 seconds in accordance with the hCG concentrations so as to sufficiently oxidize the gold colloid in the neighborhood of the working electrode.

The differential pulse voltammetry was then used to vary the potential of the working electrode in the negative direction and current changes with the potential variation were measured. The voltammetry conditions included a step pulse of 5 mV/sec and a pulse width of 25 mV The relationship between the hCG concentration of the test solution and the current value is shown in FIG. 9.

A tendency was found from FIG. 9, in which the higher the hCG concentration, the larger the current. Incidentally, when the strip immediately after the immunochromatographic analysis was observed, it was possible to visually confirm a color phenomenon on the determination part due to the accumulation of gold colloids until the hCG concentration was up to 1 ng/ml. However, when the concentration was 0.5 ng/ml or less, no visual confirmation was possible. To the contrary, according to the method of the present invention, it was confirmed that the detection of the hCG of such a low concentration as 0.1 ng/ml could also be made.

(Experiment 3)

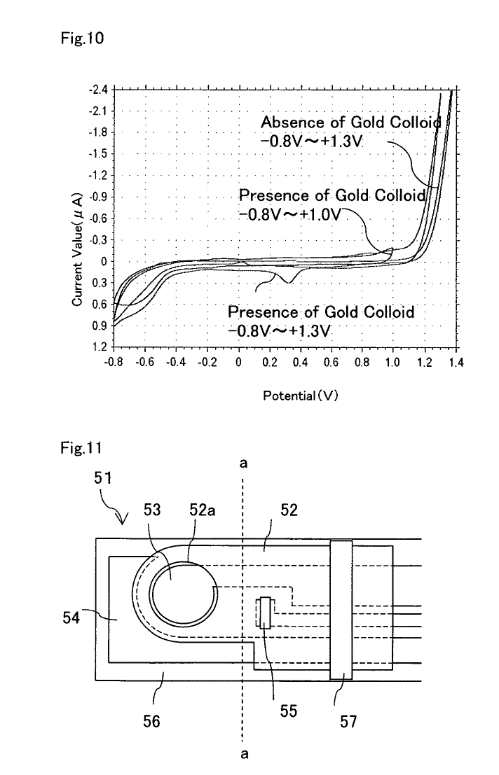

In the present experiment, it was tried to determine human Chorionic Gonadotropin (hCG) using a printed electrode having a primary antibody (anti-hCG antibody) fixed to the surface of a working electrode and an hαS antibody labeled with gold colloid in the same manner as in Experiment 1. In the present experiment, however, the cyclic voltammetry different from the differential pulse voltammetry utilized in Experiment 1 was utilized. The potential of the working electrode was set in the range of −0.8V to +1.3 V relative to the reference electrode formed of silver/silver chloride. The results obtained when using a reaction solution having an hCG concentration of 62 ng/ml were shown in FIG. 10. For comparison, the results obtained when the potential was set in the range of −0.8V to +1.0 V relative to the reference electrode formed of silver/silver chloride and the results obtained in the case where the potential was set in the range of −0.8V to +1.3 V and no antigen-antibody reaction was made were shown together in FIG. 10.

It was found from FIG. 10 that no reduced peak of gold was confirmed in the cases where the potential of the working electrode relative to silver/silver chloride reference electrode was in the range of −0.8V to +1.0 V and where the potential was in the range of −0.8V to +1.3 V and no antigen-antibody reaction was made and that the reduced peak was confirmed in the vicinity of 0.3 V in the case where the potential of the working electrode was set in the range of −0.8V to +1.3 V after an antigen-antibody reaction was made using a gold colloid-labeled anti-hαS antibody and hCG. It is understood from this fact that it is necessary to apply a potential of 1.0 V or more in oxidizing the gold colloid.

(Experiment 4)

The present experiment corresponds to the fourth embodiment. In the present experiment, a planar-type printed electrode device 51 having the shape shown in FIG. 11 and different from that used in Experiments 1 to 3 was used. The planar-type printed electrode device 51 comprises a working electrode 53 exposed to a substantially circular opening 52a formed in an insulating coat 52, a counter electrode 54 disposed so as to surround at least part of the outer circumference of the working electrode and a reference electrode 55, with all the electrodes disposed as printed on a reed-shaped insulating support substrate 56. A strap dam-structure member 57 having a surface that is more hydrophobic than the insulating film 52 is stacked on the insulating film over substantially the overall width of the printed electrode device 51 to prevent drops of a solution put on the working electrode 52 etc. from reaching a part of the device connected to a connector.

First, anti-hCG antibodies were fixed to the entire surface on one end of the printed electrode device 51 shown in FIG. 11 including the working electrode 53 and counter electrode 54, specifically to the entire surface on the left side from an a-a line in FIG. 11. The fixation of the anti-hCG antibodies and antigen-antibody reaction were performed in the same manner as in Experiment 1, provided that the hCG concentration in the present experiment was 62 ng/ml and that drops of the solution for the antigen-antibody reaction were put on the entire surface of the left side from the a-a line in FIG. 11 (hereinafter referred to as the A-surface).

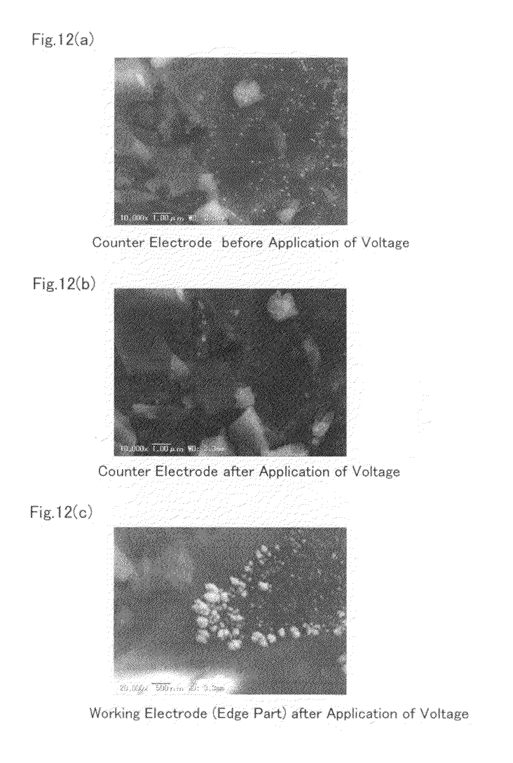

Drops of an aqueous 0.1N hydrochloric acid solution were put on the A-surface, and the potential of the working electrode was retained at −1.4 V relative to the counter electrode. The retaining time was set to be 80 seconds. The micrographs showing the surfaces of the working electrode and counter electrode assumed before and after the above operation are shown in FIG. 12.

It was confirmed from FIG. 12 that by causing the counter electrode to have a positive electrode relative to the working electrode, the gold colloid was oxidized and eluted to disappear on the surface of the counter electrode, whereas gold was deposited on the surface of the working electrode. Incidentally, the place at which the gold deposits could clearly be observed through the microscopic observation was the peripheral edge part of the working electrode.

After the deposition of gold on the surface of the working electrode, the gold deposits gathered in the neighborhood of the surface of the working electrode are electrochemically oxidized in the same manner as in Experiment 1 and current changes with the potential variation were then measured, with the potential of the working electrodes varied in the negative direction using the differential pulse voltammetry. As a result of comparing the results thus obtained with the results obtained in the case where only the surface of the working electrode was used as the reaction region in the same manner as in Experiment 1, it was confirmed that detection of higher sensitivity was realized.

(Experiment 5)

In the present experiment, the reduced peak current of gold colloid was measured in the same manner as in Experiment 1 by the use of a 0.1N hydrochloric acid solution or an aqueous saturated potassium chloride solution. The results of the cyclic voltammetry and those of the differential pulse voltammetry are shown in FIG. 13 and FIG. 14, respectively. As shown in FIG. 13, the large reduced peak current intensity is obtained in the case of using the aqueous saturated potassium chloride solution in comparison with the case of using the hydrochloric acid solution. Also, as shown in FIG. 14, the generation of noise in the vicinity of 0.1 V is confirmed in the case of the hydrochloric acid solution, whereas no noise is generated in the case of the aqueous saturated potassium chloride solution. Accordingly, it can be understood that the aqueous potassium chloride solution is more suitable as the test solution than the hydrochloric acid solution.

(Experiment 6)

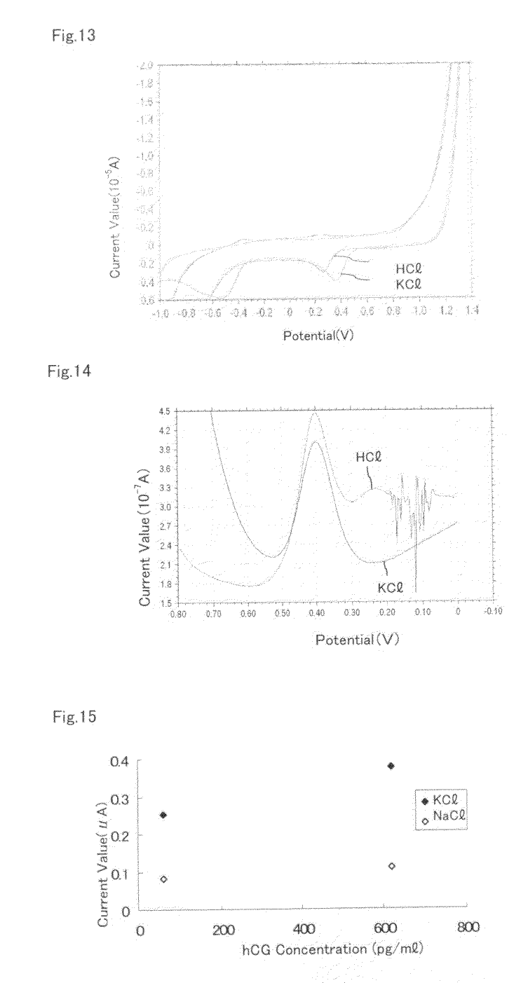

The present experiment made a study by comparison on whether which of an aqueous saturated potassium chloride solution and an aqueous 1M potassium chloride solution is suitable as a neutral solution containing chloride. To be specific, the reduced peak currents of gold colloids were measured in the same manner as in Experiment 1 using an aqueous saturated potassium chloride solution and an aqueous 1M potassium chloride solution is suitable as a neutral solution containing chloride. The results thereof are shown in FIG. 15. It was clear from the larger output obtained in the case of using the aqueous saturated potassium chloride solution that use of the aqueous saturated potassium chloride solution was preferable.

(Experiment 7)

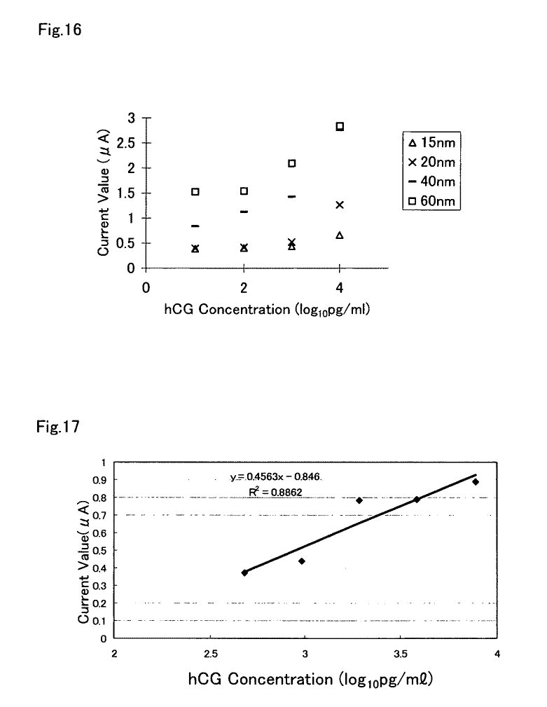

In the present experiment, studies on the optimum particle diameter were made using as metal microparticles gold colloids having particle diameters of 15 nm, 20 nm, 40 nm and 60 nm, respectively. To be specific, the characteristics of the reduced peak current of gold depending on the hCG concentration were studied in the same manner as in Experiment 1 except that colloids having particle diameters of 15 nm, 20 nm, 40 nm and 60 nm, respectively, were used as gold colloid particles used for the gold colloid-labeled hαS antibodies. The results thereof are shown in FIG. 16. The comparison of the hCG concentration characteristics revealed a tendency showing that the larger the particle diameter of the gold colloids, the larger the reduced current value and that the current value was changed up to a low concentration. However, since there is little difference in current value change between the diameters of 40 nm and 60 nm, it is expected that no discernible effect will be obtained even when the diameter of the labeled gold colloid is made larger than the diameters mentioned above. Furthermore, the reduced current value obtained when the hCG concentration was zero was 0.54 μA in the case of the gold colloid particle diameter of 80 nm, 0.2 μA in the case of the particle diameter of 40 nm and 0.14 μA in the case of the particle diameter of 15 nm. That is to say, a tendency showing that the larger the particle diameter of gold colloid particles, the larger the noise can be found. Moreover, it is understand that the diameter of 10 to 60 nm is appropriate and that diameter of 40 nm is optimum from the fact that no change in current value cannot be obtained up to the range of low concentrations when the particle diameters of the gold colloid become small.

(Experiment 8)

In the present experiment, the concentrations of hCG in biologic samples were measured using a printed electrode device having primary antibodies (anti-hCG antibodies) fixed to the surface of a working electrode. First, an analytical curve was prepared through the measurement of hCG dilution series of known concentrations made by the same method as in Experiment 1. As shown in FIG. 17, the correlation between the hcG concentrations and the current values was confirmed. Next, the current values of the test solution were measured by the same method as in Experiment 1, the hCG concentration was read from the analytical curve, and the hCG concentration of the test solution was obtained. The test solution was adjusted by diluting a urine sample with a PBS by 500 times. The results thereof are shown in Table 1. Incidentally, the hCG concentration of each sample solution was measured by the conventional ELISA method. The antibodies used in the ELISA method are the same as in Experiment 1. The results thereof are also shown in Table 1.

TABLE 1

| | Example | Conventional Method (ELISA) |
|---|---|---|
| | (ng/ml) | (ng/ml) |
| Sample 1 | 247 | 498 |
| Sample 2 | 168 | 323 |
| Sample 3 | 532 | 524 |

It is understood from Table 1 that hCG can be quantified by the present invention similarly to the ELISA that is the conventional method.

(Experiment 9)

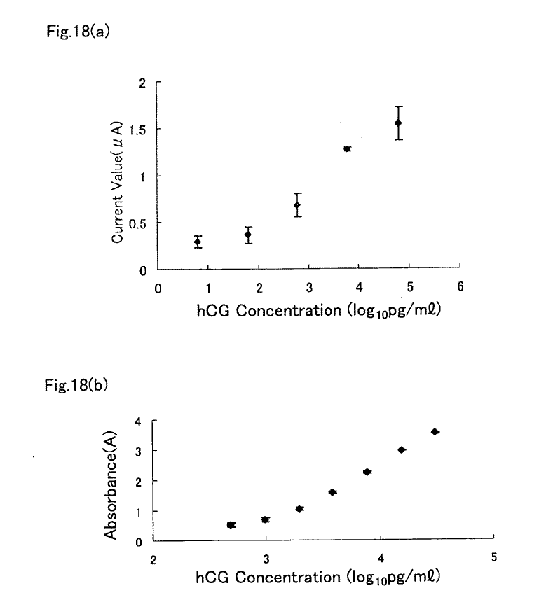

In the present experiment, the comparison was made between the determination method of the present invention and that of the ELISA method that was the conventional method using the same antigen and antibody in the two methods. The measurement method of the present invention was performed in the same manner as in Experiment 1. In the ELISA method, anti-hCG antibodies were fixed not onto an electrode but onto a plastic plate for the ELISA, an antigen-antibody reaction was made using hαS antibodies labeled not with gold colloids but with HRP (HorseRadish Peroxidase) and a TMB (3,3′,5,5′-TetraMethyl Benzidine) substrate was used in the detection reaction. The results thereof are shown in FIG. 18. As shown in FIG. 18(a), according to the method of the present invention, there was a linear relationship between the hCG concentrations and the measurement results up to around the hCG concentration of 102 pg/ml. On the other hand, as shown in FIG. 18(b), the range for obtaining the linear relationship in the ELISA method was around the hCG concentration of 103 pg/ml. Therefore, according to the determination method of the present invention, it is found that the enhancement by around ten times can be expected as compared with the ELISA method. Furthermore, while 100 μl of the sample was required, 2 μl of the sample solution that is around one fiftieth would suffice in the measurement method of the present invention. Therefore, it can be understood that the amount of the sample can be reduced to a great extent as compared with the conventional method.

(Experiment 10)

In the present experiment, noises generated between the case of measuring the electrochemical reduced peak current and the case of measuring the oxidation peak current were compared.

First, the printed electrode device having the shape shown in FIG. 11 was prepared, and antibodies were fixed to the surface of the working electrode in the same manner as in Experiment 1 using anti-hCG antigen solutions having concentrations of 13 μg/ml, 130 μg/ml, 135 μg/ml and 550 μg/ml. Additionally, as a comparative example, a printed electrode device for noise evaluation that was subjected to blocking without fixing any anti-hCG antibody thereto was prepared.

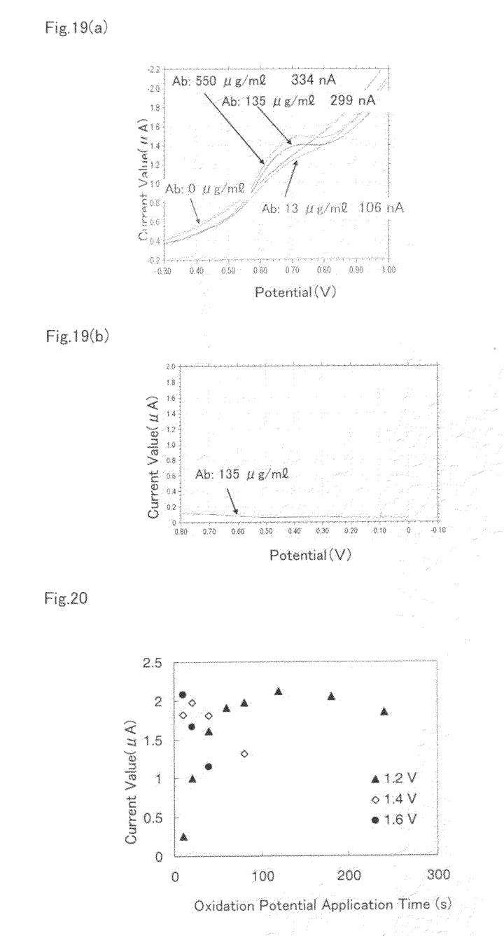

Next, the current values obtained by applying the potential to the working electrode in the positive direction were measured. The results obtained on the side oxidized are shown in FIG. 19(a). On the other hand, the current values obtained by applying the potential to the working electrode in the negative direction were measured. The results obtained on the side reduced are shown in FIG. 19(b). When focusing on the measurement results on the oxidized side in FIG. 19(a), oxidized peaks of tyrosine and triptophan contained in the antibody were confirmed in the neighborhood of the oxidized peak of gold (in the vicinity of 0.9 V). In proportion as the amount of the antibody fixed was increased to 13 μg/ml, 135 μg/ml and 550 μg/ml, the current values thereof were increased to 106 nA, 299 nA and 334 nA. These currents result from the oxidation of the tyrosine and triptophan contained in the protein or antibody used for blocking. As was clear from the measurement results on the reduced side shown in FIG. 19(b), however, no peak resulting from the antibody or protein could be confirmed in the neighborhood of the reduced peak of gold (0.3 to 0.4V). It can be understood from these results that the reduced currents measured can suppress a noise influence and a false detection possibility as compared with the oxidized currents.

(Experiment 11)

In the present experiment, the voltage applied in oxidizing and eluting metal microparticles gathered in the neighborhood of the working electrode (pretreatment voltage) was studied.

First, the printed electrode device having the shape shown in FIG. 11 was prepared and, by means of an antigen-antibody reaction, gold microparticles were gathered in the neighborhood of the surface of a working electrode. Subsequently, drops of an aqueous 0.1N hydrochloric acid solution were put on the electrode surface, and the potential of the working electrode was retained at 1.2 V, 1.4 V and 1.6 V for a prescribed period of time (0 to 200 seconds).

Next, the differential pulse voltammetry was used to vary the potential of the working electrode from 0.8 mV to 0 V, and current changes with the potential variation were measured. The voltammetry conditions were the same as before. The relationship between the oxidized potential-applying time and the current peak value with the reduction of gold observed in the vicinity of 0.3 V is shown in FIG. 20.

In FIG. 20, it was possible to observe the current peak with the reduction when the potential was set to be 1.2 V or more. When the potential was set to be less than 1.2 V, no peak of the reduced current could be confirmed in the vicinity of 0.3 V. On the other hand, a tendency showing that the higher the potential, the shorter the application time required was found. It is expected from the tendency that in the case where the oxidation potential becomes larger than 1.6 V, a slight difference in application time induces a large difference in current value. In order to secure the stability in the detection, therefore, it is necessary to set the oxidation potential to be 1.6 V or less. It was confirmed from these results that the potential for oxidizing and eluting the metal microparticles was preferably in the range of 1.2 V to 1.6 V.

(Experiment 12)

In the present experiment, the voltage application time for oxidizing and eluting the metal microparticles gathered in the neighborhood of the surface of the working electrode (pretreatment time) was studied.

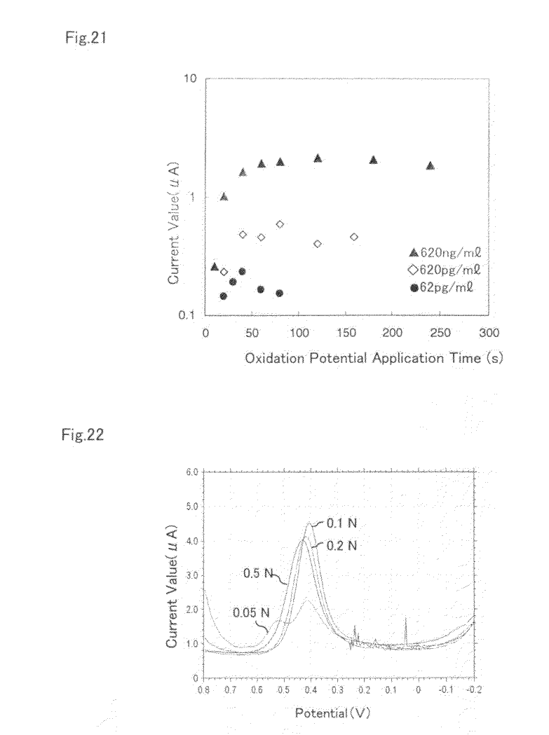

Current changes with the potential variation were measure in the same manner as in Experiment 11 except that the potential for oxidizing the metal microparticles was set to be 1.2 V and that the concentrations of hCG in the test solution were set to be 62 pg/ml, 620 pg/ml and 62 ng/ml. The results thereof are shown in FIG. 21. In FIG. 21, though measurement could be made in the application time range of around one second to 300 seconds, no discernible change in current value could be found even when the application time was set to be 100 seconds or more. It is therefore understood that the application time is preferably in the range of one second to 100 seconds. Further, when the time of applying the oxidation potential was 40 seconds or more, sufficiently high current values were obtained at all the hCG concentrations. It is understood from this fact that the application time in the range of 40 seconds to 100 seconds is particularly preferable.

(Experiment 13)

Concentration conditions of a hydrochloric acid solution that was a measurement solution for re-depositing metal particles that had been gathered in the neighborhood of the surface of the working electrode, oxidized and eluted were studied.

Plural electrodes having the same amount of gold colloid particles fixed thereto in the same manner as in Experiment 1 were prepared. The gold colloid particles were oxidized at 1.2 V for 40 seconds with 0.05N, 0.1N, 0.2N, 0.5N and 1.0N (not shown in the graph of FIG. 22) hydrochloric acid solutions, respectively, and then the differential pulse voltammetry was used to measure reduced currents with the potential variation. The results thereof are shown in FIG. 22. It is understood from FIG. 22 that the shape of the graph at the concentration of 0.05N is distorted and that the peak current value is lowered. At other concentrations including 1.0N, the results showed the similar waveforms in spite of the different peak potentials. Furthermore, since handling is difficult to perform when the concentration is unduly high, it can be understood that a 0.05N to 2N hydrochloric acid solution is appropriate and that a 0.1N to 0.5N hydrochloric acid is particularly preferable.

1. A method for determining a test substance using metal microparticles as labeled substances, comprising the steps of:
gathering in a neighborhood of a surface of a working electrode the metal microparticles in an amount corresponding to an amount of the test substance contained in a test solution;
oxidizing the metal microparticles electrochemically;
measuring a value of a current induced by electrochemically reducing the oxidized metal microparticles; and
determining a presence or absence or a concentration of the test substance based on the current value.

2. A method for determining a test substance according to claim 1, wherein the step of oxidizing the metal microparticles electrochemically comprises retaining a potential of the working electrode at a level at which the metal microparticles are oxidized electrochemically.

3. A method for determining a test substance according to claim 2, wherein the potential of the working electrode relative to a silver/silver chloride reference electrode is retained at +1.2 V to +1.6 V when oxidizing the metal microparticles electrochemically.

4. A method for determining a test substance according to claim 2, wherein the potential of the working electrode is retained for one second or more and 100 seconds or less.

5. A method for determining a test substance according to claim 1, wherein the step of oxidizing the metal microparticles electrochemically comprises varying a potential of the working electrode with time to a level at which the metal microparticles are oxidized electrochemically.

6. A method for determining a test substance according to claim 1, wherein the step of oxidizing the metal microparticles electrochemically includes controlling a potential of the working electrode in an acidic solution.

7. A method for determining a test substance according to claim 6, wherein the acidic solution is a 0.05N to 2N hydrochloric acid solution.

8. A method for determining a test substance according to claim 1, wherein the step of oxidizing the metal microparticles electrochemically includes controlling a potential of the working electrodes in a neutral solution containing chlorine.

9. A method for determining a test substance according to claim 8, wherein the neutral solution containing chlorine is an aqueous KCl solution.

10. A method for determining a test substance according to claim 1, wherein the metal microparticles are gold microparticles having a particle diameter of 10 nm to 60 nm.

11. A method for determining a test substance according to claim 1, wherein the step of oxidizing the metal microparticles electrochemically includes retaining a potential of the working electrode relative to a silver/silver chloride reference electrode at +1.2 V to +1.6 V in a 0.1N to 0.5N hydrochloric acid solution.

12. A method for determining a test substance according to claim 1, wherein the step of gathering in a neighborhood of a surface of a working electrode the metal microparticles in an amount corresponding to an amount of the test substance contained in a test solution comprises fixing to the working electrode a first binding substance that is specifically bound to the test substance, labeling with the metal microparticles a second binding substance that is specifically bound to the test substance to constitute a labeled body, supplying onto the surface of the working electrode the test solution and the labeled body to react with each other.

13. A method for determining a test substance according to claim 1, wherein the step of gathering in a neighborhood of a surface of a working electrode the metal microparticles in an amount corresponding to an amount of the test substance contained in a test solution comprises labeling with the metal microparticles a second binding substance that is specifically bound to the test substance to constitute a labeled body, fixing to at least the working electrode and a counter electrode a first binding substance that is specifically bound to the test substance, and supplying onto surfaces of the working electrode and counter electrode the test solution and the labeled body to react with each other, thereby gathering the metal microparticles in the neighborhood of the surfaces of the working electrode and counter electrode,
and the step of oxidizing the metal microparticles electrochemically includes subjecting the working electrode to potential control so as to cause the counter electrode to have a positive potential, thereby electrochemically oxidizing metal deposited on the surface of the working electrode and the metal microparticles gathered in the neighborhood of the surface of the working electrode.

14. A method for determining a test substance according to claim 1, wherein the step of gathering in a neighborhood of a surface of a working electrode the metal microparticles in an amount corresponding to an amount of the test substance contained in a test solution comprises preparing magnetic microparticles having fixed thereto a first binding substance that is specifically bound to the test substance and a labeled body having a second binding substance, which is specifically bound to the test substance, labeled with the metal microparticles, mixing the test solution, magnetic microparticles and labeled body to react with one another, and gathering the magnetic microparticles in the vicinity of the surface of the working electrode.

15. A method for determining a test substance according to claim 1, wherein the step of gathering in a neighborhood of a surface of a working electrode the metal microparticles in an amount corresponding to an amount of the test substance contained in a test solution comprises preparing an immunochromatography strip having a first binding substance, which is specifically bound to the test substance, fixed to a prescribed fixed region of the strip and a labeled body having a second binding substance, which is specifically bound to the test substance, labeled with the metal microparticles, developing the test solution and labeled body onto the immunochromatography strip, and causing the fixed region of the strip and the working electrode to overlap each other.

16. A method for determining a test substance according to claim 12, wherein the first and second binding substances are antibodies.
